(19) United States
(12) Patent Application Publication
(10) Pub. No.: US 2008/0119830 A1
(43) Pub. Date: May 22, 2008

(54) Disposable tip for laser handpiece

(76) Inventors: Paul O. Ramstad, San Jose, CA (US); Albert C. Zakowski, San Jose, CA (US); Stephen Derek Smithson, Redwood City, CA (US); Andrey Degtyaryov, Sunnyvale, CA (US); Michael Robert Gluszczak, San Jose, CA (US); Raymond Gordon White, Saratoga, CA (US); Ronald S. Bader, Boulder Creek, CA (US); Daniel K. Childs, Chicago, IL (US)

Correspondence Address: Pearl Cohen Zedek Latzer, LLP, 1500 Broadway, 12th Floor, New York, NY, 10036, US

(21) Appl. No.: 11/589,799

(22) Filed: Oct. 31, 2006

(51) Int. Cl. A61B 18/18

(52) U.S. Cl. 606/13

(57) ABSTRACT

The present invention generally relates to a disposable insert for a laser handpiece providing a hygienic surface for applying monochromatic light to a patient. The disposable insert is configured for providing vacuum to a laser cavity and provides a measure of safety to the user by insuring that the laser will be inoperative in the absence of the insert.

### BACKGROUND OF THE INVENTION

1. Field of the Invention

The present invention generally relates to a disposable insert for a laser handpiece. Specifically, the insert provides a hygienic surface for applying monochromatic light to a patient. In particular, the disposable insert is configured for providing vacuum to a laser cavity. More specifically, the insert provides a measure of safety to the user by insuring that the laser will be inoperative in the absence of the insert. By having such an insert, a laser handpiece can apply monochromatic light to a patient with heretofore unrealized results.

2. Description of the Related Art

Laser handpieces for applying electromagnetic radiation to a patient's skin for a wide variety of treatments have been long known. Patients may have many different types of skin disorders, some of which may be contagious. There is therefore a great need in the skin treatment field for devices which avoid infecting patients when the same device is used on many patients. Further, in the laser treatment field specifically, there is a great need for safety. In particular, a user of a laser treatment device does not want the laser to fire if there is some component of the device improperly installed. Having a laser misfire could cause injury to the user, the patient, or an assistant standing nearby. There is therefore a great need in the art for maintaining hygienic practices in the skin treatment field as well as providing a measure of safety to a patient. Accordingly, there is now provided with this invention an improved laser radiation treatment system which effectively overcomes the aforementioned difficulties and longstanding problems inherent in skin treatment systems. These problems have been solved in a simple, convenient, and highly effective way by which to have a disposable hygiene shield installed into a laser radiation handpiece.

### SUMMARY OF THE INVENTION

According to one aspect of the invention, a device for insertion into a handpiece comprises a body having a cavity. The body has at least one channel for providing communication between said cavity and the handpiece. The body may also have at least one means for allowing the determination of a correctly inserted device into the handpiece.

According to another aspect of the invention, a device for insertion into a handpiece is disclosed comprising a body having a base and side walls depending from the base for forming a cavity therein. The body has a pair of channels for providing communication between said cavity and the handpiece. The body also has a pair of pins extending from the body into the handpiece for allowing the determination of a correctly inserted device.

According to another aspect of the invention, a system disclosed for applying laser radiation to skin of a patient, comprising a handpiece including a laser radiation source for applying radiation into a cavity therein. The system also includes a removable body adapted for fitting into the handpiece cavity. The body has a pair of channels, wherein at least one of the channels is for providing vacuum between the skin and the handpiece. The body further includes a pair of pins extending from the body into the handpiece so that the pins can be detected therein when the body is fitted into said handpiece.

As will be appreciated by those persons skilled in the art, a major advantage provided by the present invention is to have a disposable hygienic shield in a laser handpiece in which proper installation is detected. Additional objects of the present invention will become apparent from the following description.

The method and apparatus of the present invention will be better understood by reference to the following detailed discussion of specific embodiments and the attached figures which illustrate and exemplify such embodiments.

### DESCRIPTION OF THE DRAWINGS

A specific embodiment of the present invention will be described with reference to the following drawings, wherein:

FIG. 1 is a diagrammatic view of an embodiment of the insert of the present invention and an embodiment of the laser handpiece into which it is inserted.

FIG. 2 is an orthogonal view of an embodiment of the insert of the present invention.

FIG. 3 is another orthogonal view of an embodiment of the insert of the present invention showing filters and O-rings in an exploded view.

FIG. 4 is a bottom view of an embodiment of the insert of the present invention.

FIG. 5 is a side view of an embodiment of the insert of the present invention taken along axis A-A of FIG. 3.

FIG. 6 is a side view of an embodiment of the insert of the present invention taken along axis B-B of FIG. 3.

FIG. 7 is an electrical schematic of an embodiment of a portion of the handpiece of the present invention.

### Description OF THE PREFERRED EMBODIMENT

The following preferred embodiment as exemplified by the drawings is illustrative of the invention and is not intended to limit the invention as encompassed by the claims of this application. A device adapted for insertion into a laser handpiece is disclosed herein.

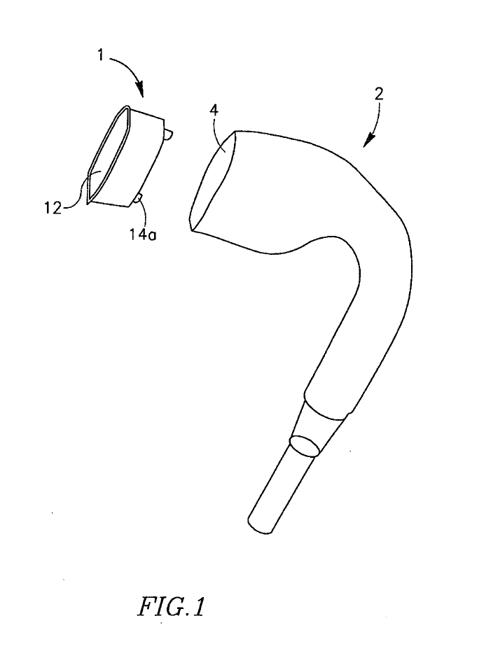

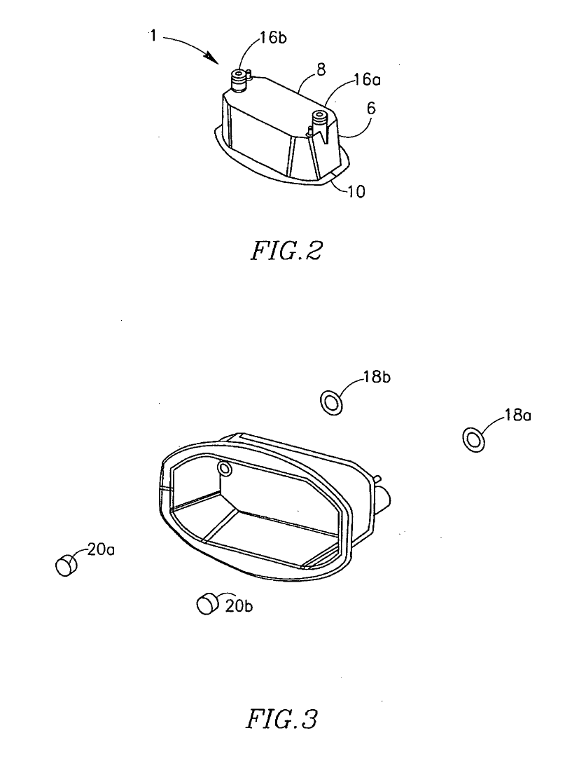

The insert 1, as illustrated generally in FIGS. 1-6 is for fitting into a laser handpiece 2. The insert acts, at least in part, as a hygiene shield for one time use on a patient. The laser handpiece has a cavity 4 into which the insert is configured for fitting. The cavity is adapted for applying a vacuum when the handpiece is applied to the skin for treatment. Laser light of particular wavelength and energy is directed to the skin while the skin is under vacuum with the handpiece in a predetermined controlled manner for selected skin treatment.

The insert 1 has an outer wall 6 which conforms to the cavity 4 of the handpiece. Typically, the thickness of the wall is in the range of from about 0.875 mm to about 1.125 mm. The insert has a base 8 which acts as a window through which the laser beam is directed. Typically, the thickness of the base is in the range of from about 0.875 mm to about 1.125 mm. Accordingly, at least the base is manufactured of a material optically transparent to the particular laser wavelength being applied. Preferably, the entire insert is made of the same material (for example, a polycarbonate having 88% light transmission and 1% haze at 2.5 mm) because laser light is reflected and redirected throughout the handpiece cavity by the walls of the cavity and thus also through the outer wall 6 of the insert. Preferably, the insert may be formed of clear GE Lexan HF 1140 Polycarbonate, FDA tripartite (ISO i0993-1 mod)/USP Class VI compliant, UL94V-2; or clear GE Lexan HP1, FDA food contact compliant, UL94V-2.

The base may also include optical properties either applied to or as part of its structure. Such optical properties may include, for example, a diffraction pattern, a refractive property, or an optical coating. Of course, as is well known to those skilled in the art the base may be made of a different thickness or optical properties than the walls of the insert.

A further alternative to the body is to include either permanent or removable reflective surfaces along at least a portion of the side walls of the insert. The reflective surface, which may extend throughout the interior or exterior surfaces of the insert cavity and which may be either a coating or a removable insertion may be made of, or a substrate coated with, gold and/or other reflective metals and/or non-metals. The reflective surfaces may be specular, diffuse, or somewhere in between.

The insert due to its exposure to laser energy necessitates the use of a material with at least a Flame Class Rating of UL 94V-2 or (3) CSA. Since the insert comes in contact with the patient's skin, a biocompatible or FDA food contact compliant material is also needed. In addition to conforming plastics, glass is also a suitable material.

The outer wall extends from the base 8 to a peripheral flange 10 which extends around the periphery of the insert. The flange 10 is preferably conformable to the skin of the patient. It is preferable that the flange not extend beyond the handpiece body, except for a portion that is exposed to the side relief of the handpiece body for easing finger access for removal of the insert. Having the flange conform to the outer profile of the handpiece body allows the insert to remain in place when the handpiece is removed from a pocket into which it is preferably stored and calibrated. The outer wall thus forms a cavity 12 within the insert itself. The flange 10 is that portion of the insert that contacts the skin of the patient and forms a sufficient seal so that a vacuum, for example, ranging from about 11 in. Hg (37 kPa.) to about 28 in. Hg (95 kPa.), can be formed therewith. The vacuum thus formed preferably draws the skin into the cavity 12 of the insert before each pulse of electromagnetic radiation is applied and releases the skin upon completion of the pulse.

A channel 14a is formed into the insert for allowing vacuum communication between the handpiece and the cavity 12 of the insert. Preferably, such a channel is formed using a tubular member 16a extending from the base. It is further preferable that another similarly shaped member 16b be symmetrically formed on the base thereby forming another symmetrically placed channel 14b. It is still further preferable that only one of the two channels be operative at any one time. For example, although the insert mates exactly with the cavity 4 of the handpiece, and the two tubular members 16a and 16b fit into mating slots within the handpiece, only one of the members (for example, 16a) may be connected to a vacuum pump. Therefore, only one channel (for example, 14a) would allow passage for a vacuum to be applied to the cavity 12 of the insert when a seal is formed by the flange with the skin. The other tubular member (for example, 16b) would preferably fit into an inoperative channel of the handpiece (not shown). (Of course, as is well known in the art, both channels may alternatively be used for drawing a vacuum either simultaneously or in a sequence.)

In order to make a tight, leak-free sealing engagement with the vacuum channel of the handpiece, it may be preferable to include O-rings 18a and 18b on the tubular members 16a and 16b, respectively. The o-rings are preferably made of elastomeric material as is well known to those skilled in the art. It is further preferable that a 70-durometer silicone elastomer be used. Alternately, other durometers and other elastomers, including Buna-N, Viton, and other rubber equivalents may also be used.

The compression of the o-rings between the sealing surfaces of the handpiece and insert provides sufficient frictional resistance to secure the insert within the handpiece cavity during use. Other latching means such as mating protrusions and depressions integral to the insert and handpiece (or components within the handpiece) could also be used for this purpose. The body may also include a latching mechanism for securing the insert into the handpiece.

It may be preferable to include particle filters 20a and 20b inserted within the channels 14a and 14b, respectively. The preferred filter material is hydrophobic polyethylene, 3 mm thick, 45-90 micron pore size. Other suitable filter materials include polypropylene, Nylon, Teflon, glass, sintered metals and non-flammable grades of cellulose, preferably Porex X-4904 High Density Polyethylene meeting FDA 21 CFR 177.1520. Materials in different thicknesses, such as a woven or matted fabric or a wire mesh, with other pore sizes may also be acceptable for use in this application. Alternatively features such as a series of closely spaced protrusions or an array of small holes in a membrane-like element spanning the vacuum channel could be integrated into the design of the insert itself to replace the particle filters described in the current invention. The addition of the filters into their respective channels may help to protect the vacuum system from clogging with skin debris and other detritus that may be produced during the skin treatment procedure.

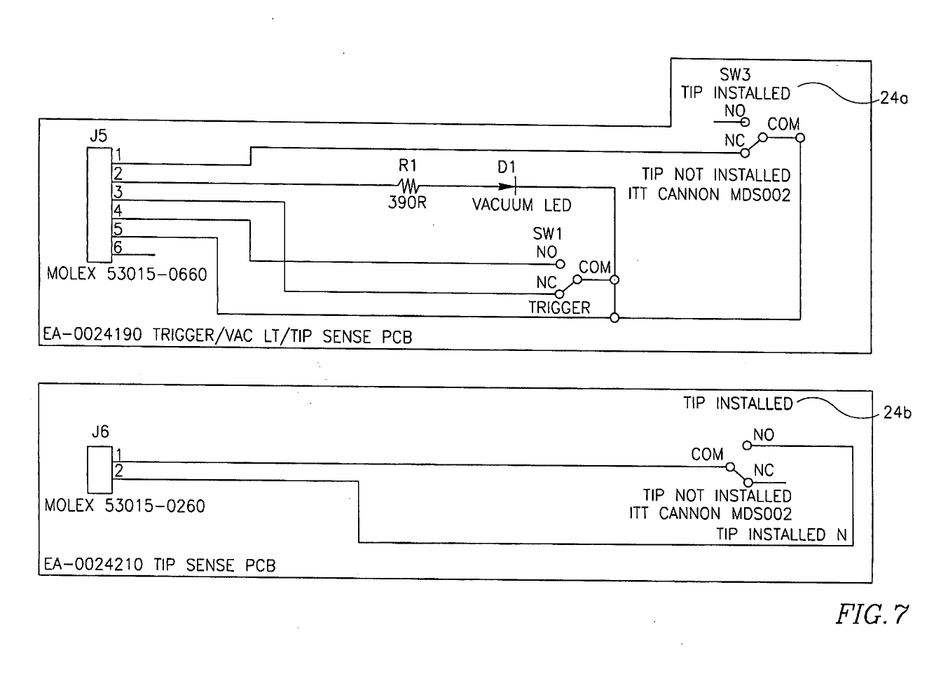

Tip pins 22a and 22b may be symmetrically placed extending from the base of the insert. These pins may be formed to matingly insert into pin receiving holes formed in the cavity of the handpiece. Within the pin receiving holes of the handpiece, which are formed to receive and engage with the tip pins 22a and 22b, are pin detectors for detecting the presence of both of the pins. The detection of the presence of both of the tip pins allows an additional level of safety for the operator and for the patient. Of course, as is well known in the art, alternatively, only one of the pin receiving holes may have a pin detector. A further alternative may exclude pins entirely and merely detect the presence of the tubular extensions 16a and 16b when they are inserted into their corresponding channels in the handpiece. Such detection of the tubular channels for conducting a vacuum would be in accordance with the electrical schematic diagram discussed with respect to FIG. 7.

FIG. 7 shows an electrical schematic diagram of an embodiment of pin detection. By providing this type of pin detection, a safety interlock with the laser is effected. As illustrated, one pin, for example 22a, may fit into a pin-receiving hole of the handpiece in which a normally closed detection switch is located. The other pin, for example, 22b, may correspondingly fit into another pin receiving hole of the handpiece in which a normally open detection switch is located. When the insert is properly secured into the handpiece, both of the pins must engage with their respective detection switches in order to allow the laser to become operative. In this particular embodiment described and illustrated in FIG. 7, when pin 22a matingly engages with its receiving hole, a pin detection switch 24a is moved from its normally closed position to an open position. Correspondingly, when pin 22b matingly engages with its receiving hole, a pin detection switch 24b is moved from its normally open position to a closed position. When the circuit in which switch 24a is broken and when the circuit in which 24b is completed are both detected, the safety interlock allows the laser to fire. Only when both circuits are detected, with switch 24a being open and switch 24b being closed, is the laser allowed to fire. This arrangement is preferable because it ensures that misdetection will not result in allowing the laser to fire.

Alternatively, the detection switches may be differently arranged. For example, switches 24a and 24b may both be normally closed switches. As a further alternative, switches 24a and 24b may both be normally open switches.

A further alternative to having pins extending from the insert into receiving holes of the insert may be to have a pin or pins in the handpiece extend into receiving holes or receiving depressions in the insert. In such an embodiment, the safety interlock would determine whether or not the pins extend from the handpiece in the correct manner. In such an embodiment, only when the pins correctly extend from the handpiece into their respective corresponding receptors in the insert, would the laser be allowed to fire.

A still further embodiment of the present invention may include using a conductive material to complete a circuit between two electrodes in the handpiece cavity. All or a selected portion of the insert may be conductive. Thus, when the insert in properly placed in the handpiece, a circuit is completed for establishing proper fit. Alternatively, the pins 22a and 22b may be conducting elements or electrodes.

A yet further embodiment of the present invention may include a capacitive or optical proximity detector for non-contact sensing of the insert. An additional embodiment may further include a means for detecting an optical signature of the base. Such detection of the optical signature of the base may include, for example, detecting the reflection signature or the absorption signature of the base for an indication of correct insertion of the tip into the handpiece.

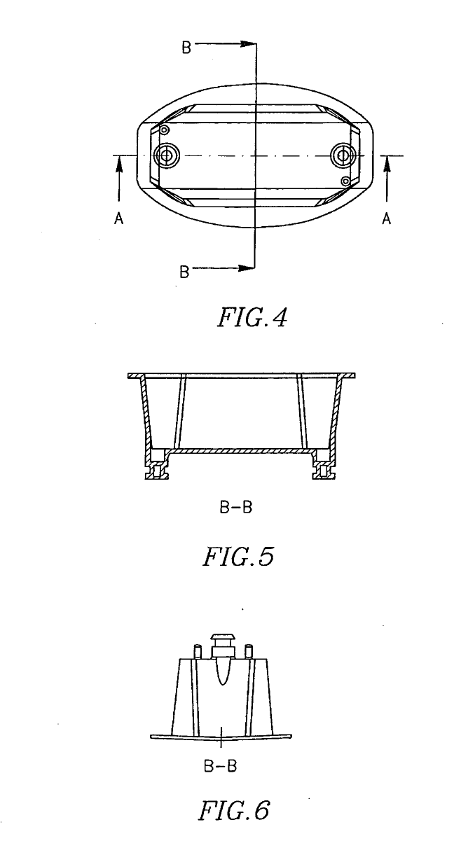

It is preferable that the insert be symmetrical about axes A-A and B-B of FIG. 4. In this way, the insert can initially be placed in the cavity of the handpiece easily. Further, since an embodiment of the present invention may have only one of the two channels (for example, 14a) operative for drawing a vacuum, if one filter, for example 20a becomes clogged, the same insert can be easily and quickly repositioned in the handpiece thereby using the other channel (for example, 14b) with the unused filter, for example, 20b. After the insert has been used on a patient, it is disposed of so that the next patient may have a new hygienic insert installed for his use. Reuse of a cleaned sterilized insert is also within the scope of this invention.

Although the particular embodiments shown and described above will prove to be useful in many applications in the skin treatment arts and laser applications in general, to which the present invention pertains, further modifications of the present invention will occur to persons skilled in the art. All such modifications are deemed to be within the scope and spirit of the present invention as defined by the appended claims.

What is claimed is:

1. A device for insertion into a handpiece, comprising:
a. a body having a cavity therein, wherein the body has at least one channel for providing communication between said cavity and the handpiece; and
b. at least one means for allowing the determination of a correctly inserted device into the handpiece.

2. The device of claim 1, wherein said determining means includes a pair of pins, wherein the correctly inserted device enables the determination of both of said pair of pins in the handpiece.

3. The device of claim 2, wherein at least one of said pair of pins is for completing a normally open circuit for allowing the determination of a correctly inserted device.

4. The device of claim 2, wherein at least one of said pair of pins is for opening a normally closed circuit for allowing the determination of a correctly inserted device.

5. The device of claim 2, wherein one of said pair of pins is for completing a normally open circuit and the other of said pair of pins opens a normally closed circuit for allowing the determination of a correctly inserted device.

6. The device of claim 2, wherein said pair of pins are for completing a normally open circuit for allowing the determination of a correctly inserted device.

7. The device of claim 2, wherein said pair of pins are for opening a normally closed circuit for allowing the determination of a correctly inserted device.

8. The device of claim 2, wherein the pins are electrodes.

9. The device of claim 1, wherein said means is for completing at least one circuit for allowing the determination of a correctly inserted device into the handpiece.

10. The device of claim 1, wherein said means includes a receptor for receiving an extension from the handpiece.

11. The device of claim 1, including a pair of channels for providing communication between said cavity and the handpiece.

12. The device of claim 1, wherein said means includes said channel.

13. The device of claim 1, wherein the body includes an optical element.

14. The device of claim 13, wherein the optical element includes a lens.

15. The device of claim 13, wherein the optical element includes a diffraction grating or pattern.

16. The device of claim 13, wherein the optical element includes a refractive element.

17. The device of claim 13, wherein the optical element includes an optical coating.

18. The device of claim 1, wherein the body includes a reflecting surface.

19. The device of claim 1, wherein the body includes a latching mechanism.

20. The device of claim 5, including a pair of channels for providing communication between said cavity and the handpiece, wherein each of said pair of channels is adapted for including a filter.

21. The device of claim 20, wherein the pins and the channels are symmetric about an axis of the device.

22. The device of claim 1, wherein said means includes a detector for detecting an optical signature of said body.

23. A device for insertion into a handpiece, comprising:
a. a body having a base and side walls depending from said base for forming a cavity therein, wherein the body has a pair of channels for providing communication between said cavity and the handpiece; and
b. a pair of pins extending from the body into the handpiece for allowing the determination of a correctly inserted device.

24. The device of claim 23, including a pair of channels for providing communication between said cavity and the handpiece.

25. The device of claim 24, wherein each of said channels is adapted for receiving a filter.

26. The device of claim 24, wherein the correctly inserted device enables the determination of both of said pair of pins in the handpiece.

27. The device of claim 24, wherein one of said pair of pins is for completing a normally open circuit and the other of said pair of pins opens a normally closed circuit for allowing the determination of a correctly inserted device.

28. The device of claim 24, wherein the pins and the channels are symmetric about an axis of the device.

29. The system of claim 23, wherein the body includes an optical element.

30. The system of claim 29, wherein the optical element includes a lens.

31. The system of claim 29, wherein the optical element includes a diffraction grating.

32. The system of claim 29, wherein the optical element includes a refractive element.

33. The system of claim 29, wherein the optical element includes an optical coating.

34. The system of claim 23, wherein the body includes a reflecting surface.

35. The system of claim 23, wherein the body includes a latching mechanism.

36. A system for applying laser radiation to skin of a patient, comprising:
a. a handpiece including a laser radiation source for applying radiation into a cavity therein; and
b. a removable body adapted for fitting into said handpiece cavity, wherein the body has a pair of channels, wherein at least one of said channels is for providing vacuum between the skin and the handpiece, and wherein said body includes a pair of pins extending from said body into said handpiece so that said pins can be detected therein when said body is fitted into said handpiece.

37. The system of claim 36, wherein, one of said pair of pins is for completing a normally open circuit and the other of said pair of pins opens a normally closed circuit for allowing the determination of a correctly inserted device.

38. The system of claim 37, wherein the body includes an optical element.

39. The system of claim 38 wherein the optical element includes a lens.

40. The system of claim 38, wherein the optical element includes a diffraction grating.

41. The system of claim 38, wherein the optical element includes a refractive element.

42. The system of claim 38, wherein the optical element includes an optical coating.

43. The system of claim 36, wherein the body includes a reflecting surface.

44. The system of claim 36, wherein the body includes a latching mechanism.
